(19) United States
(12) Patent Application Publication
(10) Pub. No.: US 2014/0276654 A1
(43) Pub. Date: Sep. 18, 2014

(54) Nasal Suction Device

(71) Applicant: ACCLARENT, INC., Menlo Park, CA (US)

(72) Inventor: Thomas R. Jenkins, Alameda, CA (US)

(73) Assignee: ACCLARENT, INC., Menlo Park, CA (US)

(21) Appl. No.: 13/832,180

(22) Filed: Mar. 15, 2013

(51) Int. Cl. A61M 1/00

(52) U.S. Cl. CPC A61M 1/0023
USPC 604/540

(57) ABSTRACT

An apparatus comprises a body, a base, and a conduit. The body comprises an absorbent material. The base comprises a non-absorbent material. The conduit is in fluid communication with the body. The conduit has a suction opening positioned within the absorbent material. The conduit is operable to draw fluid away from the absorbent material. The apparatus may be positioned within the posterior choanae or the nasopharynx of a patient. The apparatus may prevent fluids from draining from the nasal cavity into the patient's throat. In some instances, the apparatus is used as a plug during a sinus irrigation procedure to prevent irrigation fluid from traveling down the patient's throat.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Menlo Park, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein. An example of such an endoscope is the Acclarent Cyclops™ Multi-Angle Endoscope by Acclarent, Inc. of Menlo Park, Calif.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire maybe provided in accordance with the teachings of U.S. Pub. No. 2012/0078118, entitled “Sinus Illumination Lightwire Device,” published Mar. 29, 2012, the disclosure of which is incorporated by reference herein. An example of such an illuminating guidewire is the Relieva Luma Sentry™ Sinus Illumination System by Acclarent, Inc. of Menlo Park, Calif.

In addition to or in lieu of dilating ostia of sinuses, it may be desirable in some instances to irrigate the sinuses to flush contents from the sinuses. For instance, it may be desirable to provide such irrigation for therapeutic purposes and/or in order to clear a site before and/or after a sinuplasty procedure as described herein. Sinus irrigation may be performed using an irrigation catheter that is positioned within the sinus. The irrigation catheter may have one or more distally positioned openings that are configured to emit fluid (e.g., saline) as the fluid is communicated through the irrigation catheter. Examples of devices that may be used to provide sinus irrigation are the Relieva Vortex® and Relieva Vortex® 2 Sinus Irrigation Catheters by Acclarent, Inc. of Menlo Park, Calif. While procedures such as sinus irrigation are performed, it may be desirable to prevent the irrigation fluid and/or flushed debris from traveling down the patient's nasopharynx. This may be attempted using suction devices, occluding devices, and/or other kinds of devices. A variety of devices that may be used to prevent such travel are described in U.S. Pub. No. 2012/0245419, entitled “Devices, Systems and Methods for Diagnosing and Treating Sinusitis and Other Disorders of the Ears, Nose and/or Throat,” published Sep. 27, 2012, the disclosure of which is incorporated by reference herein.

While several instruments and procedures have been made and used for treatment of anatomical passageways in a patient, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side elevational view of an exemplary dilation catheter system;

FIG. 2 depicts a side elevational view of an exemplary illuminating guidewire suitable for use with the dilation catheter system of FIG. 1;

FIG. 3 depicts a side cross-sectional view of the illuminating guidewire of FIG. 2;

FIG. 4 depicts a perspective view of an exemplary endoscope suitable for use with the dilation catheter system of FIG. 1;

FIG. 5 depicts a side elevational view of the distal end of the endoscope of FIG. 5, showing an exemplary range of viewing angles;

FIG. 6 depicts a side elevational view of an exemplary irrigation catheter that may be used with the dilation catheter system of FIG. 1;

FIG. 7 depicts a side elevational view of an exemplary suction device that may be used with the irrigation catheter of FIG. 6;

FIG. 8 depicts a side elevational view of another exemplary suction device that may be used with the irrigation catheter of FIG. 6; and

FIG. 9 depicts a perspective view of yet another exemplary suction device that may be used with the irrigation catheter of FIG. 6.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

FIG. 1 shows an exemplary dilation catheter system (10) that may be used to dilate the ostium of a paranasal sinus; or to dilate some other anatomical passageway (e.g., within the ear, nose, or throat, etc.). Dilation catheter system (10) of this example comprises a dilation catheter (20), a guide catheter (30), an inflator (40), and a guidewire (50). By way of example only, dilation catheter system (10) may be configured in accordance with at least some of the teachings of U.S. Patent Pub. No. 2011/0004057, the disclosure of which is incorporated by reference herein. In some versions, at least part of dilation catheter system (10) is configured similar to the Relieva® Spin Balloon Sinuplasty™ System by Acclarent, Inc. of Menlo Park, Calif.

The distal end of dilation catheter (20) includes an inflatable dilator (22). The proximal end of dilation catheter (20) includes a grip (24), which has a lateral port (26) and an open proximal end (28). Dilation catheter (20) includes a first lumen (not shown) that provides fluid communication between lateral port (26) and the interior of dilator (22). Dilator catheter (20) also includes a second lumen (not shown) that extends from open proximal end (28) to an open distal end that is distal to dilator (22). This second lumen is configured to slidably receive guidewire (50). The first and second lumens of dilator catheter (20) are fluidly isolated from each other. Thus, dilator (22) may be selectively inflated and deflated by communicating fluid along the first lumen via lateral port (26) while guidewire (50) is positioned within the second lumen. In some versions, dilator catheter (20) is configured similar to the Relieva Ultirra™ Sinus Balloon Catheter by Acclarent, Inc. of Menlo Park, Calif. In some other versions, dilator catheter (20) is configured similar to the Relieva Solo Pro™ Sinus Balloon Catheter by Acclarent, Inc. of Menlo Park, Calif. Other suitable forms that dilator catheter (20) may take will be apparent to those of ordinary skill in the art in view of the teachings herein.

Guide catheter (30) of the present example includes a bent distal end (32) and a grip (34) at its proximal end. Grip (34) has an open proximal end (36). Guide catheter (30) defines a lumen that is configured to slidably receive catheter (20), such that guide catheter (30) may guide dilator (22) out through bent distal end (32). In some versions, guide catheter (30) is configured similar to the Relieva Flex™ Sinus Guide Catheter by Acclarent, Inc. of Menlo Park, Calif. Other suitable forms that guide catheter (30) may take will be apparent to those of ordinary skill in the art in view of the teachings herein.

Inflator (40) of the present example comprises a barrel (42) that is configured to hold fluid and a plunger (44) that is configured to reciprocate relative to barrel (42) to selectively discharge fluid from (or draw fluid into) barrel (42). Barrel (42) is fluidly coupled with lateral port (26) via a flexible tube (46). Thus, inflator (40) is operable to add fluid to dilator (22) or withdraw fluid from dilator (22) by translating plunger (44) relative to barrel (42). In the present example, the fluid communicated by inflator (40) comprises saline, though it should be understood that any other suitable fluid may be used. In some versions, inflator (40) is configured in accordance with at least some of the teachings of U.S. Pat. App. No. 61/725,523, entitled “Inflator for Dilation of Anatomical Passageway,” filed Nov. 13, 2012, the disclosure of which is incorporated by reference herein. Other suitable forms that inflator (40) may take will be apparent to those of ordinary skill in the art in view of the teachings herein.

As best seen in FIGS. 2-3, guidewire (50) of the present example comprises a coil (52) positioned about a core wire (54). An illumination wire (56) extends along the interior of core wire (54) and terminates in an atraumatic lens (58). A connector (55) at the proximal end of guidewire (50) enables optical coupling between illumination wire (56) and a light source (not shown). Illumination wire (56) may comprise one or more optical fibers. Lens (58) is configured to project light when illumination wire (56) is illuminated by the light source, such that illumination wire (56) transmits light from the light source to the lens (58). In some versions, the distal end of guidewire (50) is more flexible than the proximal end of guidewire (50). Guidewire (50) has a length enabling the distal end of guidewire (50) to be positioned distal to dilator (22) while the proximal end of guidewire (50) is positioned proximal to grip (24). Guidewire (50) may include indicia along at least part of its length (e.g., the proximal portion) to provide the operator with visual feedback indicating the depth of insertion of guidewire (50) relative to dilation catheter (20). By way of example only, guidewire (50) may be configured in accordance with at least some of the teachings of U.S. Pub. No. 2012/0078118, the disclosure of which is incorporated by reference herein. In some versions, guidewire (50) is configured similar to the Relieva Luma Sentry™ Sinus Illumination System by Acclarent, Inc. of Menlo Park, Calif. Other suitable forms that guidewire (50) may take will be apparent to those of ordinary skill in the art in view of the teachings herein.

In an exemplary dilation procedure, guide catheter (30) may first be positioned near the targeted anatomical passageway, such as a sinus ostium (O). Dilator (22) and the distal end of guidewire (50) may be positioned within or proximal to bent distal end (32) of guide catheter (30) at this stage. Guide catheter (30) is initially inserted into the nose of the patient and is advanced to a position that is within or near the ostium (O) to be dilated. This positioning of guide catheter (30) may be performed under visualization provided by an endoscope such as endoscope (60) described below. After guide catheter (30) has been positioned, the operator may advance guidewire (50) distally through guide catheter (30) such that a distal portion of the guidewire (50) passes through the sinus ostium (O) and into the sinus cavity. The operator may illuminate illumination wire (56) and lens (58), which may provide transcutaneous illumination through the patient's face to enable the operator to visually confirm positioning of the distal end of guidewire (50) with relative ease.

With guide catheter (30) and guidewire (50) suitably positioned, dilation catheter (20) is advanced along guidewire (50) and through bent distal end (32) of guide catheter (30), with dilator (22) in a non-dilated state until dilator (22) is positioned within the sinus ostium (O) (or some other targeted anatomical passageway). After dilator (22) has been positioned within the ostium (O), dilator (22) may be inflated, thereby dilating the ostium. To inflate dilator (22), plunger (44) may be actuated to push saline from barrel (42) of inflator (40) through dilation catheter (20) into dilator (22). The transfer of fluid expands dilator (22) to an expanded state to open or dilate the ostium (O), such as by remodeling the bone, etc., forming ostium (O). By way of example only, dilator (22) may be inflated to a volume sized to achieve about 10 to about 12 atmospheres. Dilator (22) may be held at this volume for a few seconds to sufficiently open the ostium (O) (or other targeted anatomical passageway). Dilator (22) may then be returned to a non-expanded state by reversing plunger (44) of inflator (40) to bring the saline back to inflator (40). Dilator (22) may be repeatedly inflated and deflated in different ostia and/or other targeted anatomical passageways. Thereafter, dilation catheter (20), guidewire (50), and guide catheter (30) may be removed from the patient.

II. Overview of Exemplary Endoscope

As noted above, an endoscope (60) may be used to provide visualization within an anatomical passageway (e.g., within the nasal cavity, etc.) during a process of using dilation catheter system (10). As shown in FIGS. 4-5, endoscope of the present example comprises a body (62) and a rigid shaft (64) extending distally from body (62). The distal end of shaft (64) includes a curved transparent window (66). A plurality of rod lenses and light transmitting fibers may extend along the length of shaft (64). A lens is positioned at the distal end of the rod lenses and a swing prism is positioned between the lens and window (66). The swing prism is pivotable about an axis that is transverse to the longitudinal axis of shaft (64). The swing prism defines a line of sight that pivots with the swing prism. The line of sight defines a viewing angle relative to the longitudinal axis of shaft (64). This line of sight may pivot from approximately 0 degrees to approximately 120 degrees, from approximately 10 degrees to approximately 90 degrees, or within any other suitable range. The swing prism and window (66) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples.

Body (62) of the present example includes a light post (70), an eyepiece (72), a rotation dial (74), and a pivot dial (76). Light post (70) is in communication with the light transmitting fibers in shaft (64) and is configured to couple with a source of light, to thereby illuminate the site in the patient distal to window (66). Eyepiece (72) is configured to provide visualization of the view captured through window (66) via the optics of endoscope (60). It should be understood that a visualization system (e.g., camera and display screen, etc.) may be coupled with eyepiece (72) to provide visualization of the view captured through window (66) via the optics of endoscope (60). Rotation dial (74) is configured to rotate shaft (64) relative to body (62) about the longitudinal axis of shaft (64). It should be understood that such rotation may be carried out even while the swing prism is pivoted such that the line of sight is non-parallel with the longitudinal axis of shaft (64). Pivot dial (76) is coupled with the swing prism and is thereby operable to pivot the swing prism about the transverse pivot axis. Indicia (78) on body (62) provide visual feedback indicating the viewing angle. Various suitable components and arrangements that may be used to couple rotation dial (74) with the swing prism will be apparent to those of ordinary skill in the art in view of the teachings herein.

By way of example only, endoscope (60) may be configured in accordance with at least some of the teachings of U.S. Pub. No. 2010/0030031, the disclosure of which is incorporated by reference herein. In some versions, endoscope (60) is configured similar to the Acclarent Cyclops™ Multi-Angle Endoscope by Acclarent, Inc. of Menlo Park, Calif. Other suitable forms that endoscope (60) may take will be apparent to those of ordinary skill in the art in view of the teachings herein.

III. Exemplary Sinus Irrigation Catheter

As noted above, it may be desirable in some instances to irrigate the sinuses to flush contents from the sinuses. FIG. 6 shows an exemplary irrigation catheter (80) that may be used to perform sinus irrigation. Irrigation catheter (80) of this example includes a flexible tubular member (82) that has a distal opening (84), a plurality of transverse openings (86) just proximal to distal opening (84), and a proximal fitting (88). Proximal fitting (88) is configured as a standard luer fitting that may be coupled with any suitable fluid source (e.g., a syringe filled with saline, etc.). Fluid communicated through proximal fitting (88) will travel along tubular member (82) and exit via openings (84, 86).

Irrigation catheter (80) of this example further includes a support assembly (90) comprising a support tube (92) and a grip (94). Tubular member (82) is slidably disposed within support assembly (90). Support tube (90) has greater rigidity than tubular member (82) such that support tube (90) provides structural support for tubular member (82) as tubular member (82) is being positioned in a patient. Tubular member (82) may also include indicia along at least part of its length in order to provide visual feedback relating to the depth of insertion of tubular member (82). By way of example only, irrigation catheter (80) may be configured similar to the Relieva Vortex® and Relieva Vortex® 2 Sinus Irrigation Catheters by Acclarent, Inc. of Menlo Park, Calif. Other suitable forms that irrigation catheter (80) may take will be apparent to those of ordinary skill in the art in view of the teachings herein.

Tubular member (82) of the present example is sized for insertion through guide catheter (30). In particular, tubular member (82) may be inserted through guide catheter (30) before dilation catheter (20) has been inserted in guide catheter (30) or after dilation catheter (20) has been removed from guide catheter (30). It should be understood that irrigation catheter (80) may also be advanced along guidewire (50), if desired. In some uses, irrigation catheter (80) is advanced through guide catheter (30) such that openings (84, 86) are positioned near a desired irrigation/flush site. Endoscope (60) and/or illumination from guidewire (50) may be used to provide visualization during the positioning of irrigation catheter (80). Once irrigation catheter (80) is suitably positioned, fluid is communicated along tubular member (82) from proximal fitting (88) to openings (84, 86), such that the fluid is sprayed from openings (85, 86) at the irrigation/flush site. This irrigation procedure may be performed before dilation catheter (20) is used to dilate the ostia (O) and/or after dilation catheter (20) is used to dilate the ostia (O). In some instances, irrigation catheter (80) is used when no dilation procedure is being performed. Various other settings in which irrigation catheter (80) may be used will be apparent to those of ordinary skill in the art in view of the teachings herein.

It should be understood that an irrigation process using irrigation catheter (80) may result in the need for fluid and/or other debris to drain from the irrigation/flush site. Otherwise, the patient may gag, cough, choke, or otherwise experience discomfort. In some instances, the operator may simply have the patient lean forward such that the fluid drains from the patient's nose. An occlusion device may be positioned within the patient's nasopharynx, in the posterior choanae, or elsewhere to prevent the fluid from draining down the patient's throat. In addition or in the alternative, a suction device may be used to draw away the drainage. Additional devices that may be used to handle drainage from an irrigation process will be described in greater detail below; while still other examples will be apparent to those of ordinary skill in the art in view of the teachings herein.

IV. Exemplary Nasal Suction Device

FIG. 7 shows an exemplary suction device (100) that may be used to handle drainage from an irrigation procedure performed using irrigation catheter (80) or using some other irrigation device. Suction device (100) of this example comprises an absorbent body (110) secured to a base (120). Body (110) and base (120) are sized to be positioned within the posterior choanae or the nasopharynx of a patient. A suction tube (130) is secured to base (120) by a pair of flanges (132, 134), which provide a fluid tight seal at the interface between suction tube (130) and base (120). Suction tube (130) has an open distal end (136). The proximal end of suction tube (130) may be coupled with a suction source (e.g., vacuum pump, vacuum wall outlet, syringe, etc.), such that suction tube (130) may provide suction at open distal end (136). Open distal end (136) is positioned within body (110).

Body (110) is configured to absorb fluids and is also pliable such that body (110) does not cause trauma to the tissue that comes in contact with body (110). In some versions, body (110) is configured to expand as it absorbs fluid, such that body (110) will at least slightly bear outwardly on the inner wall of the anatomical passageway in which body (110) is disposed. This may enable body (110) to generally conform to the shape of the anatomical passageway and avoid fluids leaking past body (110). In some versions, body (110) comprises foam. Various suitable materials and structures that may be used to form body (110) will be apparent to those of ordinary skill in the art in view of the teachings herein.

Base (120) is also pliable and atraumatic, yet base (120) is formed of a non-absorbent material that will provide a fluid tight seal such that the fluid will not pass through or around base (120) when base (120) is properly positioned within an anatomical passageway such as the posterior choanae or the nasopharynx. In some versions, base (120) comprises a membrane. Base (120) may also resiliently bear against the wall of the anatomical passageway, such that base (120) conforms to the shape of the anatomical passageway without providing fluid drainage paths between the outer perimeter of base (120) and the wall of the anatomical passageway. While base (120) is shown as only making up a relatively small portion of the overall thickness of suction device (100), it should be understood that body (110) and base (120) may have any other suitable relative thicknesses. Various suitable materials that may be used to form base (120) will also be apparent to those of ordinary skill in the art in view of the teachings herein.

In an exemplary use, suction device (100) is positioned in a patient's posterior choanae or nasopharynx while irrigation catheter (80) is positioned at the irrigation site in the patient's nasal cavity. Suction device (100) is positioned such that body (110) is generally directed toward the patient's nasal cavity while base (120) is generally directed toward the patient's throat. In this example, suction tube (130) exits through the underside of base (120) and is fed along the nasopharynx and out the patient's mouth. Irrigation fluid is then communicated through irrigation catheter (80) while suction is communicated to suction tube (130). The irrigation fluid drains through the patient's nasal cavity and into body (110), which absorbs the fluid. As body (110) absorbs the fluid, suction tube (130) draws the fluid out of body (110) via open distal end (136). This prevents body (110) from becoming oversaturated to the point where suction device (100) no longer effectively plugs the drainage path. The configuration and absorbance of body (110) prevents fluid from flowing quickly past suction tube (130).

FIG. 8 shows another exemplary suction device (200) that may be used to handle drainage from an irrigation procedure performed using irrigation catheter (80) or using some other irrigation device. Suction device (200) of this example is substantially similar to suction device (100) in that suction device (200) comprises an absorbent body (210) secured to a base (220) and a suction tube (230). Absorbent body (210) may be configured an operable just like absorbent body (110) described above. Similarly, base (220) may be configured and operable just like base (120) described above. However, in this example, suction tube (230) enters the top side of body (210) and is secured to base (220) by a plurality of tethers (232). In some other versions, tube (230) is bonded to body (210); and base (220) is bonded to body (210) such that tube (230) is bonded to tube (230) via body (210). Other suitable ways in which body (210), base (220), and body (210) may be secured will be apparent to those of ordinary skill in the art in view of the teachings herein. In the present example, suction tube (230) still also has an open distal end (234) and may be coupled with a suction source at the proximal end of suction tube (230).

In an exemplary use, suction device (200) is positioned in a patient's posterior choanae or nasopharynx while irrigation catheter (80) is positioned at the irrigation site. Suction device (200) is positioned such that body (210) is generally directed toward the patient's nasal cavity while base (220) is generally directed toward the patient's throat. In this example, suction tube (230) exits through the patient's nostril. Irrigation fluid is then communicated through irrigation catheter (80) while suction is communicated to suction tube (230). The irrigation fluid drains through the patient's nasal cavity and into body (210), which absorbs the fluid. As body (210) absorbs the fluid, suction tube (230) draws the fluid out of body (210) via open distal end (234). This prevents body (210) from becoming oversaturated to the point where suction device (200) no longer effectively plugs the drainage path.

While suction device (100) has a suction tube (130) exiting the bottom of suction device (100), and suction device (200) has suction tube (130) exiting the top of suction device (200), it should be understood that either suction device (100, 200) may be modified to have its suction tube (130, 230) exit the side of suction device (100, 200) or have some other relationship with body (110, 210).

FIG. 9 shows yet another exemplary suction device (300) that may be used to handle drainage from an irrigation procedure performed using irrigation catheter (80) or using some other irrigation device. Suction device (300) of this example is substantially similar to suction devices (100, 200) in that suction device (300) comprises an absorbent body (310) secured to a base (320) and a suction tube (330). Absorbent body (310) may be configured and operable just like absorbent body (110) described above. Similarly, base (320) may be configured and operable just like base (120) described above. In addition, a flange (332) secures suction tube (330) to base (320). However, in this example, the portion of suction tube (330) within body (310) comprises a main trunk portion (334) having a plurality of outwardly extending branches (340). Main trunk portion (334) also includes a distal opening (336) and a plurality of transverse openings (338), all of which are in fluid communication with the interior of suction tube (330).

Branches (340) include distal openings (342) that are also in fluid communication with the interior of suction tube (330). It should be understood that openings (336, 338, 342) provide additional points of suction within body (310), which may assist in drawing fluid from body (310) more effectively than versions having just one suction opening within body (310). It should also be understood that branches (340) may include transverse openings (338), if desired. Furthermore, main trunk portion (334) may lack transverse openings (338). In some other versions, main trunk portion (334) is substantially omitted, such that all branches (340) terminate at a common stump or other kind of interface near flange (332) and extend upwardly and outwardly from the interface. Various suitable configurations and arrangements of branches (340) will be apparent to those of ordinary skill in the art in view of the teachings herein.

Suction device (300) may be used just like suction device (100) described above, including routing suction tube (330) along the nasopharynx and out the patient's mouth. Alternatively, suction tube (330) may exit the top of body (310) similar to suction tube (230) or may have any other suitable relationship with body (310).

It should be understood that with the effective seals provided by suction devices (100, 200, 300), an operator may perform irrigation procedures using a fluid pressure that is substantially higher than the fluid pressure that the operator might otherwise use in cases where a suction wand or other device were the only device used to handle draining irrigation fluid, etc. The substantially higher irrigation fluid pressures enabled by suction devices (100, 200, 300) may result in better cleaning of the sinuses by the irrigation fluid.

While suction devices (100, 200, 300) have been described above as being used in the context of an irrigation procedure, it should be understood that suction devices (100, 200, 300) may be used in a variety of other procedures. Similarly, it should be understood that suction devices (100, 200, 300) may be used to absorb and suction fluids other than irrigation fluid. By way of example only, suction devices (100, 200, 300) may be used to absorb medication (e.g., lidocaine, etc.), blood, water, and/or any other fluid that may otherwise drain through the nasopharynx.

IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a body, wherein the body comprises an absorbent material;
(b) a base, wherein the base is secured to the body, wherein the base comprises a non-absorbent material; and
(c) a conduit in fluid communication with the body, wherein the conduit has a suction opening positioned within the absorbent material, wherein the conduit is operable to draw fluid away from the absorbent material.

2. The apparatus of claim 1, wherein the conduit passes through the base.

3. The apparatus of claim 2, wherein the conduit is secured to the base by one or more flanges.

4. The apparatus of claim 1, wherein the body has a first side and a second side, wherein the base is secured to the second side.

5. The apparatus of claim 4, wherein the conduit passes through the first side of the body.

6. The apparatus of claim 5, further comprising a plurality of tethers securing the conduit to the base.

7. The apparatus of claim 1, wherein the absorbent material of the body is configured to swell in response to fluid.

8. The apparatus of claim 1, wherein the base is formed of a resilient material.

9. The apparatus of claim 1, wherein the body and the base are dimensioned to fit in an anatomical passageway of a patient.

10. The apparatus of claim 1, wherein the conduit includes a plurality of branches.

11. The apparatus of claim 10, wherein the branches are positioned within the body,

12. The apparatus of claim 10, wherein the branches are in fluid communication with the conduit.

13. The apparatus of claim 12, wherein each branch of the plurality of branches has at least one suction opening in fluid communication with the conduit, wherein the at least one suction opening of each branch is positioned within the absorbent material.

14. The apparatus of claim 10, wherein the conduit further comprises a trunk portion, wherein the branches extend outwardly from the trunk portion.

15. The apparatus of claim 14, wherein the trunk portion has a plurality of suction openings positioned within the absorbent material.

16. An apparatus comprising:
(a) a body, wherein the body comprises an absorbent material;
(b) a base, wherein the base is secured to the body, wherein the base comprises a non-absorbent material; and
(c) a plurality of conduits positioned within the body, wherein each conduit has at least one suction opening positioned within the absorbent material, wherein the conduit is configured to draw fluid away from the absorbent material via the suction openings.

17. A method of using a suction device in a patient to handle fluid from a fluid source, wherein the suction device comprises an absorbent body secured to a non-absorbent base and a suction conduit having a suction opening positioned within the absorbent body, wherein the method comprises:
(a) positioning the suction device in an anatomical passageway of a patient, wherein the act of positioning comprises:
(i) positioning the absorbent body toward the fluid source, and
(ii) positioning the base away from the fluid source;
(b) receiving fluid in the absorbent body; and
(c) applying suction to the suction conduit, thereby drawing fluid from the absorbent body.

18. The method of claim 17, wherein the anatomical passageway comprises the posterior choanae or the nasopharynx.

19. The method of claim 17, wherein the act of positioning the suction device further comprises routing the conduit through the patient's nostril.

20. The method of claim 17, further comprising irrigating a portion of the patient's sinus cavity using an irrigation fluid, wherein the act of receiving fluid in the absorbent body comprises receiving the irrigation fluid in the absorbent body.
